(19) United States
(12) Patent Application Publication
(10) Pub. No.: US 2012/0270742 A1
(43) Pub. Date: Oct. 25, 2012

(54) Obesity Dependent Adipokine Biomarkers for Prostate Cancer

(76) Inventors: Neil E. Fleshner, Toronto (CA); Greg Trottier, Toronto (CA); Mike Connor, Richmond Hill (CA); Karen Hersey, Toronto (CA); Karen Chadwick, Toronto (CA)

(21) Appl. No.: 13/451,985

(22) Filed: Apr. 20, 2012

Related U.S. Application Data
(60) Provisional application No. 61/478,043, filed on Apr. 21, 2011.

(51) Int. Cl. C40B 30/00; G01N 33/574; C12Q 1/68

(52) U.S. Cl. 506/7; 435/6.12; 436/501

(57) ABSTRACT

There is provided herein adipokine biomarkers for prostate cancer and methods and uses thereof.

### RELATED APPLICATION

This application claims priority from U.S. Provisional Patent Application No. 61/478,043.

### FIELD OF THE INVENTION

The present invention relates to adipokine biomarkers for prostate cancer and methods and uses thereof.

### BACKGROUND

Prostate cancer is the most common cancer in American men and is the second leading cause of cancer death. The uncertainty regarding the appropriate clinical management of prostate cancer in many patients is related to an incomplete and unclear understanding of the molecular and genetic changes involved in prostate cancer development and disease progression.

A variety of clinical models or nomograms have been developed to aid clinicians with pre-treatment risk assessment. For example, since 1988, the routine use of serum prostate-specific antigen (PSA) testing in men at risk for prostate cancer has led to more favorable disease characteristics at presentation (stage migration) and earlier diagnosis and treatment. For patients newly diagnosed with prostate cancer, there are three well-defined predictors of disease extent and outcome following treatment. These factors are clinical tumor stage (T1-T4) by digital rectal examination, Gleason score of the diagnostic biopsy specimen and serum PSA level. However, each of these factors alone has not proven definitive in predicting disease severity and outcome for an individual patient. Clinical staging by digital rectal examination may underestimate the presence of extracapsular disease extension in 30-50% of patients. Although biopsy Gleason score may be helpful in predicting pathologic stage and outcome following treatment at either end of the spectrum (i.e. Gleason 2-4 or Gleason 8-10 tumors), it is not as helpful for the majority of patients who present with Gleason 5-7 disease. As risk assessment for patients newly diagnosed with prostate cancer continues to evolve, newer tools, such as genetic or molecular determinants are needed to better predict the behaviour of an individual tumor.

### SUMMARY OF THE INVENTION

In an aspect, there is provided an assay for of qualifying/predicting the likelihood of prostate cancer in a subject comprising: categorizing the subject as high BMI (>27) or low BMI (≦27); measuring the level of expression of at least one adipokine in a serum sample from the subject; correlating the level of expression of the at least one adipokine in the serum sample to a control sample; and determining that the level of expression is predictive for prostate cancer if there is exists a differential level of expression between the serum sample and the control sample; wherein the at least one adipokine is at least one of: Resistin, MCP-1 and IL-8 if the subject is low BMI; and IL-1β, IL-6 and IL-8 if the subject is high BMI.

In a further aspect, there is provided an assay for qualifying/predicting the likelihood of low-grade prostate cancer in a subject comprising: categorizing the subject as high BMI (>27) or low BMI (≦27); measuring the level of expression of at least one adipokine in a serum sample from the subject; correlating the level of expression of the at least one adipokine in the serum sample to a control sample; and determining that the level of expression is predictive for low-grade prostate cancer if there exists a differential level of expression between the serum sample and the control sample; wherein the at least one adipokine is at least one of: Resistin, PAI and IL-8 if the subject is low BMI; and IL-8, Adipo:leptin and Adiponectin (preferably IL-8) if the subject is high BMI.

In a further aspect, there is provided an assay for qualifying/predicting the likelihood of high-grade prostate cancer in a subject comprising: categorizing the subject as high BMI (>27) or low BMI (≦27); measuring the level of expression of at least one adipokine in a serum sample from the subject; correlating the level of expression of the at least one adipokine in the serum sample to a control sample; and determining that the level of expression is predictive for high-grade prostate cancer if there exists a differential level of expression between the serum sample and the control sample; wherein the at least one adipokine is at least one of: MCP-1, Adipo:leptin and Adiponectin if the subject is low BMI; and NGF, IL-1β, IL-6, HGF and Adiponectin if the subject is high BMI.

In a further aspect, there is provided a method of qualifying/predicting the likelihood of prostate cancer in a subject comprising: categorizing the subject as high BMI (>27) or low BMI (≦27); measuring the level of expression of at least one adipokine in a serum sample from the subject; and correlating the level of expression of the at least one adipokine in the serum sample to a control sample; wherein a differential level of expression of the at least one adipokine, in the subject sample compared to the control sample, is predictive for prostate cancer; and wherein the at least one adipokine is at least one of: Resistin, MCP-1 and IL-8 if the subject is low BMI; and IL-1β, IL-6 and IL-8 if the subject is high BMI.

In a further aspect, there is provided a method of qualifying/predicting the likelihood of low-grade prostate cancer in a subject comprising: categorizing the subject as high BMI (>27) or low BMI (≦27); measuring the level of expression of at least one adipokine in a serum sample from the subject; and correlating the level of expression of the at least one adipokine in the serum sample to a control sample; wherein a differential level of expression of the at least one adipokine, in the subject sample compared to the control sample, is predictive for low-grade prostate cancer; and wherein the at least one adipokine is at least one of: Resistin, PAI and IL-8 if the subject is low BMI; and IL-8, Adipo:leptin and Adiponectin if the subject is high BMI.

In a further aspect, there is provided a method of qualifying/predicting the likelihood of high-grade prostate cancer in a subject comprising: categorizing the subject as high BMI (>27) or low BMI (≦27); measuring the level of expression of at least one adipokine in a serum sample from the subject; and correlating the level of expression of the at least one adipokine in the serum sample to a control sample; wherein a differential level of expression of the at least one adipokine, in the subject sample compared to the control sample, is predictive for high-grade prostate cancer; and wherein the at least one adipokine is at least one of: MCP-1, Adipo:leptin and Adiponectin if the subject is low BMI; and NGF, IL-1β, IL-6, HGF and Adiponectin if the subject is high BMI.

In a further aspect, there is provided a system for qualifying/predicting the likelihood of prostate cancer in a subject comprising: a determination module configured to receive, in relation to a subject, an indication of high BMI (>27) or low BMI (≦27) and a serum sample, and to perform at least one analysis on the serum sample to determine the level of expression of at least one adipokine; an analysis module configured to correlate the level of expression of the at least one adipokine in the serum sample with a control sample; a display module configured to display a likelihood of prostate cancer if there exists a differential level of expression in at least one adipokine selected from: Resistin, MCP-1 and IL-8 if the subject is low BMI; and IL-1β, IL-6 and IL-8 if the subject is high BMI.

In a further aspect, there is provided a system for qualifying/predicting the likelihood of low-grade prostate cancer in a subject comprising: a determination module configured to receive, in relation to a subject, an indication of high BMI (>27) or low BMI (≦27) and a serum sample, and to perform at least one analysis on the serum sample to determine the level of expression of at least one adipokine; an analysis module configured to correlate the level of expression of the at least one adipokine in the serum sample with a control sample; a display module configured to display a likelihood of prostate cancer if there exists a differential level of expression in at least one adipokine selected from: Resistin, PAI and IL-8 if the subject is low BMI; and IL-8, Adipo:leptin and Adiponectin if the subject is high BMI.

In a further aspect, there is provided a system for qualifying/predicting the likelihood of high-grade prostate cancer in a subject comprising: a determination module configured to receive, in relation to a subject, an indication of high BMI (>27) or low BMI (≦27) and a serum sample, and to perform at least one analysis on the serum sample to determine the level of expression of at least one adipokine; an analysis module configured to correlate the level of expression of the at least one adipokine in the serum sample with a control sample; a display module configured to display a likelihood of prostate cancer if there exists a differential level of expression in at least one adipokine selected from: MCP-1, Adipo:leptin and Adiponectin if the subject is low BMI; and NGF, IL-1β, IL-6, HGF and Adiponectin if the subject is high BMI.

In a further aspect, there is provided a kit for qualifying/predicting the likelihood of prostate cancer in a subject comprising reagents for detecting at least one adipokine and instructions for carrying out the methods described herein.

### BRIEF DESCRIPTION OF THE DRAWING

These and other features of the preferred embodiments of the invention will become more apparent in the following detailed description in which reference is made to the appended drawing wherein:

FIG. 1 is a summary of which adipokines were predictive for prostate cancer, high grade (HG) prostate cancer, and low grade (LG) prostate cancer. All markers with AUCs above 0.5 and with a lower 95^{th} confidence interval above 0.5 are in bold. Those adipokines which ranked well, but had confidence intervals crossing 0.5, are in italics and not bolded.

### DETAILED DESCRIPTION

In the following description, numerous specific details are set forth to provide a thorough understanding of the invention. However, it is understood that the invention may be practiced without these specific details.

Obesity is a major problem in many parts of the world and has been linked to several different cancers. The association between obesity and increased prostate cancer (PCa) risk is controversial¹. However, evidence is stronger for a connection between obesity and increasing grade of PCa, more aggressive disease and mortality²⁻⁶. Though many mechanisms could contribute, one consideration is that hormones released from adipocytes potentiate carcinogenesis and/or growth of already established cancers.

Adipokines, defined as cytokine hormones released from adipocytes, have recently gained attention as players in metabolic syndrome, states of chronic inflammation, and several cancers^{1, 7, 8}, including prostate cancer. Leptin and adiponectin are examples of commonly studied adipokines with conflicting evidence for an association with PCa¹. Several other serum adipokines have been studied in prostate cancer, but most studies did not examine prostate cancer grade and none accounted for differences in BMI. Therefore, the relationship between many of the adipokines studies thus far still remains to be defined.

In an aspect, there is provided an assay for of qualifying/predicting the likelihood of prostate cancer in a subject comprising: categorizing the subject as high BMI (>27) or low BMI (≦27); measuring the level of expression of at least one adipokine in a serum sample from the subject; correlating the level of expression of the at least one adipokine in the serum sample to a control sample; and determining that the level of expression is predictive for prostate cancer if there exists a differential level of expression between the serum sample and the control sample; wherein the at least one adipokine is at least one of: Resistin, MCP-1 and IL-8 (preferably Resistin) if the subject is low BMI; and IL-1β, IL-6 and IL-8 if the subject is high BMI.

The term “level of expression” or “expression level” as used herein refers to a measurable level of expression of the products of biomarkers, such as, without limitation, the level of messenger RNA transcript expressed or of a specific exon or other portion of a transcript, the level of proteins or portions thereof expressed of the biomarkers, the number or presence of DNA polymorphisms of the biomarkers, the enzymatic or other activities of the biomarkers, and the level of specific metabolites.

As used herein, the term “control” or “control sample” refers to a specific value or dataset that can be used to prognose or classify the value, e.g. expression level or reference expression profile obtained from the test sample associated with an outcome class. A person skilled in the art will appreciate that the comparison between the expression of the biomarkers in the test sample and the expression of the biomarkers in the control will depend on the control used. For example, in various aspects described herein, the control is determined from serum sample(s) from healthy individuals/populations.

The term “differentially expressed” or “differential expression” as used herein refers to a difference in the level of expression of the biomarkers that can be assayed by measuring the level of expression of the products of the biomarkers, such as the difference in level of messenger RNA transcript or a portion thereof expressed or of proteins expressed of the biomarkers. In a preferred embodiment, the difference is statistically significant. The term “difference in the level of expression” refers to an increase or decrease in the measurable expression level of a given biomarker, for example as measured by the amount of messenger RNA transcript and/or the amount of protein in a sample as compared with the measurable expression level of a given biomarker in a control.

The term “sample” as used herein refers to any fluid, cell or tissue sample from a subject that can be assayed for biomarker expression products and/or a reference expression profile, e.g. genes differentially expressed in subjects.

In a further aspect, there is provided an assay for qualifying/predicting the likelihood of low-grade prostate cancer in a subject comprising: categorizing the subject as high BMI (>27) or low BMI (≦27); measuring the level of expression of at least one adipokine in a serum sample from the subject; correlating the level of expression of the at least one adipokine in the serum sample to a control sample; and determining that the level of expression is predictive for low-grade prostate cancer if there exists a differential level of expression between the serum sample and the control sample; wherein the at least one adipokine is at least one of: Resistin, PAI and IL-8 (preferably Resistin) if the subject is low BMI; and IL-8, Adipo:leptin and Adiponectin (preferably IL-8) if the subject is high BMI.

In a further aspect, there is provided an assay for qualifying/predicting the likelihood of high-grade prostate cancer in a subject comprising: categorizing the subject as high BMI (>27) or low BMI (≦27); measuring the level of expression of at least one adipokine in a serum sample from the subject; correlating the level of expression of the at least one adipokine in the serum sample to a control sample; and determining that the level of expression is predictive for high-grade prostate cancer if there exists a differential level of expression between the serum sample and the control sample; wherein the at least one adipokine is at least one of: MCP-1, Adipo:leptin and Adiponectin if the subject is low BMI; and NGF, IL-1β, IL-6, HGF and Adiponectin if the subject is high BMI.

As used herein, “low-grade prostate cancer” refers to any prostate cancer with a Gleason score equal or below 6. “High-grade prostate cancer” refers to any prostate cancer with a Gleason score equal or higher than 8.

In a further aspect, there is provided a method of qualifying/predicting the likelihood of prostate cancer in a subject comprising: categorizing the subject as high BMI (>27) or low BMI (≦27); measuring the level of expression of at least one adipokine in a serum sample from the subject; and correlating the level of expression of the at least one adipokine in the serum sample to a control sample; wherein a differential level of expression of the least one adipokine, in the subject sample compared to the control sample, is predictive for prostate cancer; and wherein the at least one adipokine is at least one of: Resistin, MCP-1 and IL-8 (preferably Resistin) if the subject is low BMI; and IL-1β, IL-6 and IL-8 if the subject is high BMI.

In a further aspect, there is provided a method of qualifying/predicting the likelihood of low-grade prostate cancer in a subject comprising: categorizing the subject as high BMI (>27) or low BMI (≦27); measuring the level of expression of at least one adipokine in a serum sample from the subject; and correlating the level of expression of the at least one adipokine in the serum sample to a control sample; wherein a differential level of expression of the least one adipokine, in the subject sample compared to the control sample, is predictive for low-grade prostate cancer; and wherein the at least one adipokine is at least one of: Resistin, PAI and IL-8 (preferably Resistin) if the subject is low BMI; and IL-8, Adipo:leptin and Adiponectin (preferably IL-8) if the subject is high BMI.

In a further aspect, there is provided a method of qualifying/predicting the likelihood of high-grade prostate cancer in a subject comprising: categorizing the subject as high BMI (>27) or low BMI (≦27); measuring the level of expression of at least one adipokine in a serum sample from the subject; and correlating the level of expression of the at least one adipokine in the serum sample to a control sample; wherein a differential level of expression of the least one adipokine, in the subject sample compared to the control sample, is predictive for high-grade prostate cancer; and wherein the at least one adipokine is at least one of: MCP-1, Adipo:leptin and Adiponectin if the subject is low BMI; and NGF, IL-1β, IL-6, HGF and Adiponectin if the subject is high BMI.

In a further aspect, there is provided a system for qualifying/predicting the likelihood of prostate cancer in a subject comprising: a determination module configured to receive, in relation to a subject, an indication of high BMI (>27) or low BMI (≦27) and a serum sample, and to perform at least one analysis on the serum sample to determine the level of expression of at least one adipokine; an analysis module configured to correlate the level of expression of the at least one adipokine in the serum sample with a control sample; a display module configured to display a likelihood of prostate cancer if there exists a differential level of expression in at least one adipokine selected from: Resistin, MCP-1 and IL-8 (preferably Resistin) if the subject is low BMI; and IL-1β, IL-6 and IL-8 if the subject is high BMI.

In a further aspect, there is provided a system for qualifying/predicting the likelihood of low-grade prostate cancer in a subject comprising: a determination module configured to receive, in relation to a subject, an indication of high BMI (>27) or low BMI (≦27) and a serum sample, and to perform at least one analysis on the serum sample to determine the level of expression of at least one adipokine; an analysis module configured to correlate the level of expression of the at least one adipokine in the serum sample with a control sample; a display module configured to display a likelihood of prostate cancer if there exists a differential level of expression in at least one adipokine selected from: Resistin, PAI and IL-8 (preferably Resistin) if the subject is low BMI; and IL-8, Adipo:leptin and Adiponectin (preferably IL-8) if the subject is high BMI.

In a further aspect, there is provided a system for qualifying/predicting the likelihood of high-grade prostate cancer in a subject comprising: a determination module configured to receive, in relation to a subject, an indication of high BMI (>27) or low BMI (≦27) and a serum sample, and to perform at least one analysis on the serum sample to determine the level of expression of at least one adipokine; an analysis module configured to correlate the level of expression of the at least one adipokine in the serum sample with a control sample; a display module configured to display a likelihood of prostate cancer if there exists a differential level of expression in at least one adipokine selected from: MCP-1, Adipo:leptin and Adiponectin if the subject is low BMI; and NGF, IL-1β, IL-6, HGF and Adiponectin if the subject is high BMI.

In a further aspect, there is provided a kit for qualifying/predicting the likelihood of prostate cancer in a subject comprising reagents for detecting at least one adipokine and instructions for carrying out the methods described herein.

In the various aspects, the differential level of expression predictive of prostate cancer is characterized by a higher level of expression in the serum sample than in the control sample.

In the various aspects, the level of expression of the at least one adipokine is preferably measured by measuring the protein level in the serum sample

A person skilled in the art will appreciate that a number of methods can be used to detect or quantify the level of protein products of the biomarkers within a sample, such as binding with antibodies, Western blot analysis and use of reporter proteins.

Similarly, a person skilled in the art will appreciate that a number of methods can be used to detect or quantify the level of RNA products of the biomarkers within a sample, including arrays, such as microarrays, RT-PCR (including quantitative RT-PCR), nuclease protection assays and Northern blot analyses. The term “nucleic acid” includes DNA and RNA and can be either double stranded or single stranded.

The term “hybridize” or “hybridizable” refers to the sequence specific non-covalent binding interaction with a complementary nucleic acid. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. For example, 6.0× sodium chloride/sodium citrate (SSC) at about 45° C., followed by a wash of 2.0×SSC at 50° C. may be employed.

The term “probe” as used herein refers to a nucleic acid sequence that will hybridize to a nucleic acid target sequence. In one example, the probe hybridizes to an RNA product of the biomarker or a nucleic acid sequence complementary thereof. The length of probe depends on the hybridization conditions and the sequences of the probe and nucleic acid target sequence. In one embodiment, the probe is at least 8, 10, 15, 20, 25, 50, 75, 100, 150, 200, 250, 400, 500 or more nucleotides in length.

Many cytokines associated with obesity (adipokines) have been implicated in prostate cancer (PCa). We sought to determine the potential of several serum adipokines to act as biomarkers for PCa. This case-control study included prostate biopsy patients derived from a serum biobank at the University of Toronto. One hundred PCa cases and 100 controls were chosen with an even mix of patients having a body mass index (BMI) above and below 27 kg/m². A panel of serum adipokines (Adiponectin, leptin, PAI, Resistin, HGF, IL-1β, IL-6, IL-8, MCP1, NGF, TNFα) were assayed and their ability to discriminate PCa outcome was evaluated by area under the receiver operating characteristic curve (AUC). We also performed a multivariable analysis and compared the accuracy of a model containing clinical variables only to a model including adipokine levels in predicting prostate cancer and high-grade prostate cancer. This patient cohort had a median PSA of 5.8 ng/ml, a median age of 62 years and a median BMI of 27.2 kg/m². Resistin (AUC 0.64) was a marker strongly discriminating cancer from no cancer among those with a BMI ≦27 kg/m². In those with a BMI >27 kg/m², IL-1β (AUC 0.61) and IL-6 (AUC 0.61) displayed the best discrimination. In the case of Gleason ≧7 disease, NGF (AUC 0.69), IL-1β (AUC 0.66), IL-6 (AUC 0.64), HGF (AUC 0.64) and adiponectin (AUC 0.64) were strongly predictive among those with a higher BMI, while no markers were strongly predictive in the lower BMI group. For Gleason 6 disease, only resistin (AUC 0.67) was strongly predictive in the low BMI patients and IL-8 (AUC 0.66) in the high BMI patients.

Our study was initiated to assess a panel of adipokines in relation to PCa diagnosis in a case-control design in patients not taking metformin or statins. Subjects were stratified based on BMI to determine if adipokine associations with PCa were dependent on obesity status.

The following example(s) are illustrative of various aspects of the invention, and do not limit the broad aspects of the invention as disclosed herein

### Examples

### Methods

### Patient Selection:

The Princess Margaret Hospital maintains an ethics-approved serum biobank of over 1500 patients undergoing prostate biopsy since September 2008 with no prior diagnosis of PCa. Patients with diabetes medications or taking HMG Co-A reductase inhibitors were excluded since these medications can alter adipokine measurements. Using a case-control design, 100 controls with no cancer, 50 cases with low grade (LG) PCa (Gleason pattern 3 only) and 50 cases with high grade (HG) PCa (Gleason pattern 4 or greater) were chosen randomly. In each group, patients were stratified so an equal number had a BMI ≦27 and >27 kg/m².

Biopsies were performed in a “for cause” fashion at the Princess Margaret Hospital in Toronto. All patients who had a first-time biopsy had a standardized 11 core technique performed by one of three uro-radiologists and those who had a repeat biopsy had a standardized 15 core technique performed¹⁸.

Serum Sample Collection and Analysis:

Patients providing consent to participate in the UHN Genitourinary (GU) BioBank had blood specimens procured prior to prostate biopsy. Serum separator tubes were kept ambient for 30 minutes following blood collection, stored at 4° C. during transport to the lab, and then promptly centrifuged at 1500×g for 15 minutes at 4° C. The serum was divided into four 500 uL aliquots in 2 mL cryovials and stored in vapour phase liquid nitrogen until use. Following research ethics board approval of the current protocol, 200 serum samples procured between September 2008 and October 2009 from selected participants. Frozen serum samples were thawed and 25 □l of the specimen was used to measure the levels of 11 proteins, including adiponectin, leptin, PAI, Resistin, HGF, IL-□, IL-6, IL-8, MCP-1, NGF and TNF-□ using Milliplex Multi-Analyte Profiling kits (Adipokine panels A and B; Millipore; Billerica, Mass., USA) in combination with a Luminex x100 System (Luminex; Austin, Tex., USA). These bead-based kits allow for the measurement of multiple analytes in a single well. Standard curves were fit to seven standard values for each adipokine and the unknown sample adipokine values were determined accordingly. Each assay was run in duplicate with two internal controls to validate the measurement consistency between and within experiments.

Statistical Analysis:

Association of clinical variables and adipokine levels versus PCa outcomes was examined with the Wilcoxon Rank Sum test for continuous variables, Fisher's Exact test for nominal variables and the Mantel Haenszel test for ordinal variables. The association between individual adipokines and BMI was determined with Spearman's correlation using BMI as a continuous variable in addition to a Chi-squared test when BMI was stratified as a categorical variable (>27 or ≦27). Associations between individual adipokines were examined with Spearman's correlation.

Area under the receiver operating characteristic curve (AUC) was used to compare how each of the individual biomarkers discriminated for overall PCa diagnosis, LG disease and HG disease. Adjusted receiver operating characteristic (AROC) curves comprising the weighted average of performance of the individual markers across the obese and non-obese stratified patient groups were use to improve precision when the pre-specified BMI groups were combined. Otherwise, separate ROC curves were generated for adipokines stratified by BMI group (>27 and ≦27) and thereby compared within the respective BMI subgroup.

Multivariate logistic regression models were implemented in order to assess the ability of clinical variables and Adipokine biomarkers to predict prostate cancer status, and also to discriminate between high grade PCa and low grade or no PCa. The area under the ROC curve (AUC) was estimated with a 95% confidence interval as a measure of the predictive ability of each model and hypothesis tests were implemented to compare the predictive ability of competing models. Models were restricted to no more than 1 parameter for every 10 events (the lesser of the number of cases and the number of controls). Predictors were selected using forward selection with alpha to enter the model set to 0.20. Analysis was performed using SAS v9.2. Statistical significance for comparing AUCs was set to alpha=0.05.

Note:
For each outcome and group of patients, we estimated AUC for 3 models:
a model based only on clinical variables (model #1)
a model based on clinical variables and Adipokine biomarkers (model #2)
a model based on the above model (clinical variables and Adipokine biomarkers), with the Adipokine biomarkers removed (model #3)
no variables were forced into any models

Results and Discussion

The clinical and pathologic demographics of the cases and control patients are displayed in Table 1. The only clinical factor assessed that was associated with increased likelihood of PCa was abnormal DRE. Prior negative biopsy was associated with a less likelihood of having PCa.

Non-parametric associations of individual adipokines with PCa in the whole cohort are presented in Table 2. Elevated IL-6 and IL-8 were associated with PCa. IL-6 and NGF were the only adipokines to selectively associate with HG disease while IL-8 was selective for LG disease. To determine if there were any interactions between different adipokines, Spearman's correlation was performed on all paired adipokine combinations showing no clear correlation between any of the tested markers (data not shown). Correlation with BMI was only found with leptin (p<0.01) and no other markers (data not shown).

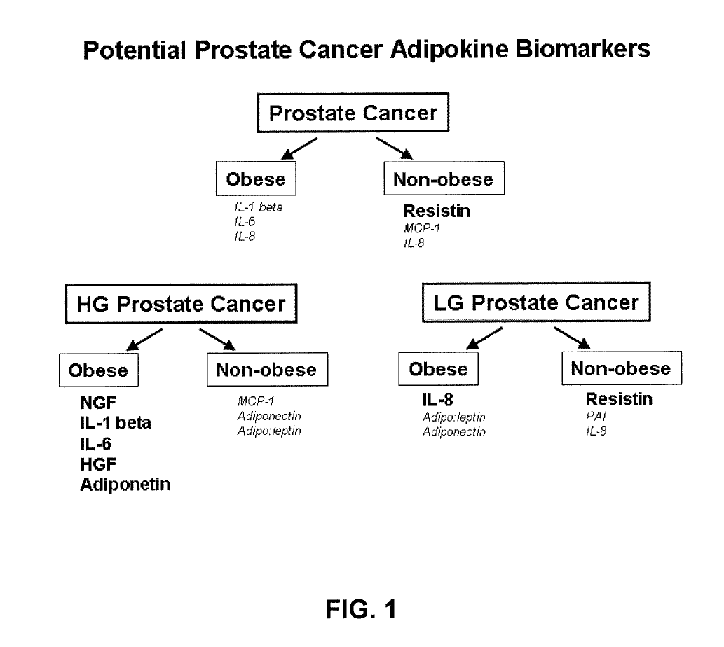

The predictive accuracy of individual adipokines in relation to PCa outcomes is shown in Table 3. Adipokines were ranked in terms of their AUC values. As used herein, “predictive” refers to those markers with AUCs greater than 0.5 and a lower 95^{th} confidence interval above 0.5. As noted in FIG. 1, other adipokines ranked well, but had confidence intervals crossing 0.5 and as such, are not described herein as “predictive”. A person skilled in the art would recognize that such adipokines would also have utility as biomarkers for prostate cancer.

For discrimination of PCa versus no PCa, IL-1, IL-6 and resistin were the best markers. NGF and IL-6 were the sole predictors for HG disease. Only IL-8 was predictive of LG PCa.

When the cohort was subdivided by the pre-specified BMI subgroups, there was a unique adipokine profile apparent for the lower and higher BMI subgroups. In the obese group (Table 4), NGF, IL-1 beta, IL-6, HGF, and adiponectin were predictive of HG PCa. NGF in particular had a considerably high AUC (0.69) for a single marker. For LG PCa discrimination, IL-8 was the only marker with predictive value. In the non-obese group (Table 5), the ranking of adipokines was completely different. Resistin was predictive for PCa and LG PCa. No markers were predictive for HG PCa in the lower BMI subgroup. A summary of the adipokines having predictive value according to BMI and PCa grade is shown in FIG. 1.

Table 6 demonstrates the 3 models to predict prostate cancer. As demonstrated Adipokine biomarkers can add to the ability to predict PCa status (AUC for the clinical model 0.68 95% CI 0.6-0.75 vs. 0.74 95% CI 0.67-0.81).

Adipokines also improve High grade PCa prediction (Table 7). The AUC for the clinical model was 0.77 95% CI 0.69-0.85) compared to 0.81 95% CI 0.74-0.88.

This study assessed the potential for several serum adipokines to predict for PCa. ROC curve analysis was chosen to determine the predictive accuracy of our marker panel based on the NCI recommendations that this is the method of choice to evaluate new biomarkers¹⁹. Our major discovery is the identification of novel adipokines with predictive accuracy specific for HG PCa in comparison to LG PCa. In addition we demonstrated that adding adipokine levels may improve the ability to predict PCa and HG PCa compared to models including only clinical variables. The other novel finding in this study is the dependency of adipokine predictive accuracy on obesity status (summarized in FIG. 1). Of interest, many of the markers we examined had much better AUC values than we have shown previously with PSA (0.55) in our patient population. These markers could have important clinical implications in the risk stratification of patients considering biopsy.

Parekh et al studied the ability of several of the same serum adipokines from our test panel (adiponectin, resistin, IL-6, NGF, HGF) to discriminate for PCa²⁰. In this case-control study assessing 54 different biomarkers in 250 men, resistin (AUC 0.56, p=0.04) and IL-6 (AUC 0.56, p=0.06) were the only markers from our test panel with predictive accuracy for PCa. However, this study did not examine different PCa grades and did not stratify by BMI. We found the same results with resistin (AUC 0.57) and IL-6 (0.59) having predictive value for PCa. However, when BMI was stratified and PCa grade was examined as an outcome in our study, several adipokines surfaced as having good predictive accuracy. We feel that BMI or some other index of obesity should be considered when assessing these markers since many of them are affected by body fat composition.

Most of our adipokine markers for PCa were found to be predictive in higher BMI and higher grade patients. This outcome coincides with the link between obesity and more advanced PCa¹⁻³. Moreover, most of the markers tested contribute to the web of pathways involved in inflammation and could explain the many inter-marker correlations we observed. There is a well-established link between obesity and inflammation⁴, which could further explain the preference of marker positivity in obese patients.

Our positive marker profile for HG disease is unique compared to LG or overall cancer, with adiponectin, HGF and IL-6 being the only markers previously assessed in HG disease. Two studies have shown that adiponectin levels were associated with advanced stage disease^{13, 14}, while one of these studies showed that it was inversely associated with HG disease in the subgroup of men that were overweight¹³. In one case-control study, no association of adiponectin or IL-6 with HG PCa was noted when BMI was included in a multivariable model⁹. However, this study was underpowered to rule out many of these markers in a multivariable analysis. In another study, IL-6 was shown to be predictive of biochemical failure after prostatectomy²¹. In summary, after controlling for BMI, IL-6 and adiponectin were predictors of HG PCa.

Previous studies have assessed serum levels of HGF in relation to localized, advanced and different grades of PCa²²⁻²⁵. Collectively, these studies have shown that HGF levels are elevated in PCa and even more so in advanced disease or higher grades. Our study is in agreement, showing graded increasing levels from controls to LG to HG PCa. None of the previous HGF studies subdivided patients by BMI. Our results show that HGF was only predictive for HG PCa in overweight patients. With a larger study it is possible that the AUC could improve for LG patients as well.

NGF and IL-1β have not been examined as serum biomarkers for HG PCa. NGF is a circulating pleiotrophic peptide with several roles including cross-talk between the nervous system and immune system²⁶. The prostate is the second most abundant source of NGF and experimental studies have shown that the pro-proliferative NGF receptor trkA becomes dominant over the pro-apoptotic p75 receptor in PCa²⁷. Our results show NGF as having the highest predictive accuracy of all markers for HG PCa in patients with a higher BMI. These results are in line with cell culture studies showing that NGF can restore androgen sensitivity of DU 145 cells²⁷ and that NGF induces expression of genes involved in progression of PCa²⁸. With regard to IL-1β previous histochemical studies showed that IL-1β expression selectively occurred in normal prostate tissue as opposed to IL-1α, which was selective for PCa tissue²⁹. In contrast, using an implanted PCa mouse model, IL-1β expression was shown to be important for angiogenesis and invasiveness of the tumor³⁰. With respect to our results, histochemical levels may not relate to serum concentrations and thereby suggests a non-prostate source of IL-1β—possibly adipose tissue. To our knowledge, our results are the first to show that serum NGF levels and IL-1β could have predictive potential for HG PCa.

For LG disease, IL-8 was predictive in the low BMI patients and resistin was predictive in the high BMI patients. Although prior evidence shows both markers to have an association with PCa, the selectivity for LG disease and the dependence on obesity status is a unique finding. PCa cell line studies showed that resistin, through the PI3 kinase pathway stimulated proliferation³¹. Immunohistochemical staining was greater in patient specimens with PCa than those with BPH³¹. A small case-control study of 68 patients showed that there was a tendency toward lower serum levels in patients with more advanced PCa³², thus supporting our selective findings in LG PCa. IL-8 levels have also been examined in a case-control fashion in an older study of 149 patients showing that IL-8 levels increased with increasing Jewett-Whitmore stage³³. Other studies with PCa tissue specimens have shown that IL-8 is also more strongly expressed in higher Gleason grade PCa³⁴ and castration-resistant PCa³⁵. Therefore, the selective discrimination of IL-8 for LG PCa in obese patients in our study is unexpected given the results from previous studies. However, previous studies did not specifically assess serum levels in relation to Gleason grade so that a direct comparison of our results to prior studies is not possible.

In summary, we found several serum adipokine biomarkers that maintained predictive accuracy for PCa in a differential pattern that depended on PCa grade and obesity status. Most of these were selective for patients with a higher BMI. Considering that the average BMI in the US and Canada is probably higher than 27, the results of this study are of particular interest. Though serum levels of adipokines vary with PCa grade and BMI, the exact source driving these adipokine levels is not known and may not reflect local expression at the prostate.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

TABLE 1
Patient cohort characteristics

| | | | Patients | | | Patients | | |
|---|---|---|---|---|---|---|---|---|
| | | | with PCa | | | with PCa | | |
| | Total cohort | | (all grades) | | | (high grade) | | |
| Variable | value | | Value | | P value | value | | P value |
| Total N (% tot) | 200 | | 100 | | | 50 | | |
| Median BMI (range) | 27.2 | (18.3-47.0) | 27.8 | (20.5-47) | 0.16 | 27.7 | (20.7-39.0) | 0.77 |
| BMI substrata; N (% row) | | | | | | | | |
| ≦27 | 95 | | 45 | (47) | | 24 | (25) | |
| >27 | 105 | | 55 | (52) | NA | 26 | (25) | NA |
| Median Age (range) | 62 | (57-68) | 63 | (58-69) | | 63 | (59-71) | |
| Family history; N (% col): | | | | | | | | |
| No | 179 | (90) | 90 | (90) | | 46 | (92) | |
| Yes | 20 | (10) | 10 | (10) | 1.0 | 4 | (8) | 0.79 |
| Ethnicity; N (% col): | | | | | | | | |
| Other | 188 | (94) | 96 | (96) | | 46 | (92) | |
| African | 9 | (6) | 2 | (2) | 0.17 | 4 | (8) | 0.24 |
| Previous negative biopsy; | | | | | | | | |
| N (% col): | | | | | | | | |
| No | 142 | (71) | 77 | (77) | | 43 | (86) | |
| Yes | 58 | (29) | 23 | (23) | 0.09 | 7 | (14) | 0.007 |
| DRE finding; N (% col): | | | | | | | | |
| Normal | 151 | (76) | 67 | (67) | | 27 | (54) | |
| Abnormal | 49 | (25) | 33 | (33) | 0.008 | 23 | (46) | <0.001 |
| Median PSA in ng/ml | 5.8 | (4.2-8.0) | 5.96 | (4.2-8.0) | 0.60 | 6.6 | (4.5-8.2) | 0.21 |
| (IQR): | | | | | | | | |

PCa = prostate cancer;
BMI = body mass index in kg/m²;
DRE = digital rectal exam;
PSA = prostate specific antigen

TABLE 2
Measured adipokine levels and association with prostate cancer

| | No Cancer | Prostate Cancer | | Low Grade Prostate | | High Grade Prostate | |
|---|---|---|---|---|---|---|---|
| | (N = 100) | (All grades; N = 100) | | Cancer (N = 50) | | Cancer (N = 50) | |
| | Median | Median | | Median | | Median | |
| | measurement | measurement | P* | measurement | P* | measurement | P |
| Marker | (IQR) | (IQR) | value | (IQR) | value | (IQR) | value* |
| Adiponectin (μg/ml) | 6.48 (3.38-10.91) | 6.68 (4.094-9.59) | 0.77 | 6.75 (4.38-9.64) | 0.67 | 6.46 (3.30-9.54) | 0.75 |
| PAI-1 (ng/ml) | 71.19 (40.06-105.38) | 68.44 (34.89-113.10) | 0.91 | 72.49 (46.38-114.22) | 0.31 | 55.80 (30.41-110.59) | 0.33 |
| Resistin (ng/ml) | 5.79 (4.01-9.53) | 7.38 (4.54-10.77) | 0.06 | 7.31 (5.01-10.88) | 0.18 | 7.57 (4.38-10.25) | 0.28 |
| HGF (ng/ml) | 1.28 (0.91-1.95) | 1.51 (0.94-2.11) | 0.18 | 1.44 (0.94-2.03) | 0.79 | 1.61 (1.00-2.12) | 0.12 |
| IL-1 β (pg/ml) | 3.18 (2.26-6.38) | 3.99 (2.28-116.00) | 0.11 | 3.63 (2.20-143.00) | 0.49 | 4.99 (2.47-107.20) | 0.12 |
| IL-6 (pg/ml) | 7.81 (6.33-11.00) | 8.82 (7.23-15.10) | 0.02 | 8.20 (6.39-16.4) | 0.59 | 9.66 (7.37-13.90) | 0.02 |
| IL-8 (pg/ml) | 8.97 (7.03-11.80) | 10.10 (7.92-13.40) | 0.04 | 11.0 (8.55-13.8) | 0.008 | 9.16 (7.74-11.90) | 0.91 |
| Leptin (ng/ml) | 15.25 (6.03-39.93) | 12.94 (4.70-32.01) | 0.53 | 13.04 (4.50-29.91) | 0.58 | 1.06 (4.94-51.62) | 0.91 |
| MCP-1 (pg/ml) | 228 (153-367) | 294 (149-483) | 0.27 | 310 (151-514) | 0.42 | 280 (161-449) | 0.53 |
| NGF (pg/ml) | 51.9 (30.30-109.00) | 53.7 (30.9-121.1) | 0.35 | 48.1 (29.1-104.9) | 0.40 | 81.6 (41.5-135.8) | 0.01 |
| TNF-α (pg/ml) | 13.3 (10.60-17.80) | 12.9 (10.6-15.6) | 0.48 | 12.6 (10.9-15.1) | 0.36 | 13.0 (10.3-16.5) | 0.92 |
| Adipo:Leptin (Ratio) | 439.8 (102.2-1600) | 549.6 (156.0-1761.0) | 0.61 | 531.0 (185.3-1560.7) | 0.52 | 514.2 (71.1-1965.3) | 0.75 |

IQR = interquartile range; PAI = plasminogen activator inhibitor; HGF = hepatocyte growth factor; IL = interleukin; MCP-1 = monocyte chemotactic protein 1; NGF = nerve growth factor, TNF = tumor necrosis factor
*Wilcoxon Rank Sum test

TABLE 3
ROC curve analysis of adipokines versus prostate cancer

| | | | | | Low Grade | |
|---|---|---|---|---|---|---|
| | | | High Grade | | Cancer | |
| | Cancer vs | | vs No High | | vs No Low | |
| | No Cancer | | Grade Cancer | | Grade Cancer | |
| Marker | Rank | AUC (95% CI) | Rank | AUC (95% CI) | Rank | AUC (95% CI) |
| Adiponectin | 9 | 0.51 (0.43-0.60) | 12 | 0.48 (0.39-0.57) | 6 | 0.49 (0.44-0.62) |
| PAI | 10 | 0.49 (0.41-0.58) | 13 | 0.45 (0.36-0.55) | 3 | 0.55 (0.45-0.64) |
| Resistin | 3 | 0.57 (0.50-0.66) | 5 | 0.55 (0.46-0.64) | 2 | 0.56 (0.48-0.65) |
| HGF | 5 | 0.55 (0.47-0.63) | 3 | 0.58 (0.48-0.67) | 9 | 0.50 (0.41-0.59) |
| IL-1 beta | 4 | 0.56 (0.48-0.64) | 4 | 0.56 (0.48-0.65) | 7 | 0.53 (0.43-0.62) |
| IL-6 | 1 | 0.59 (0.51-0.66) | 2 | 0.60 (0.51-0.69) | 8 | 0.52 (0.43-0.62) |
| IL-8 | 2 | 0.58 (0.51-0.66) | 10 | 0.49 (0.40-0.59) | 1 | 0.63 (0.54-0.72) |
| Insulin | 13 | 0.48 (0.39-0.56) | 9 | 0.51 (0.42-0.61) | 11 | 0.45 (0.36-0.55) |
| Leptin | 12 | 0.47 (0.39-0.55) | 8 | 0.51 (0.42-0.60) | 10 | 0.46 (0.36-0.55) |
| MCP-1 | 6 | 0.54 (0.47-0.63) | 6 | 0.53 (0.44-0.62) | 5 | 0.54 (0.44-0.64) |
| NGF | 7 | 0.53 (0.45-0.61) | 1 | 0.61 (0.52-0.70) | 12 | 0.45 (0.35-0.54) |
| TNF alpha | 11 | 0.47 (0.38-0.54) | 7 | 0.51 (0.41-0.60) | 13 | 0.45 (0.36-0.53) |
| Adipo:Lep | 8 | 0.53 (0.45-0.61) | 11 | 0.48 (0.39-0.57) | 8 | 0.53 (0.46-0.63) |

AUC = area under the receiver operating characteristic curve;
PAI = plasminogen activator inhibitor;
HGF = hepatocyte growth factor;
IL = interleukin;
MCP-1 = monocyte chemotactic protein 1;
NGF = nerve growth factor,
TNF = tumor necrosis factor

TABLE 4
ROC curve analysis of adipokines versus prostate
cancer in patients with a BMI ≧ 27 Kg/m²

| | | | High Grade Cancer vs No | | Low Grade Cancer vs No | |
|---|---|---|---|---|---|---|
| | Cancer versus No Cancer | | High Grade Cancer | | Low Grade Cancer | |
| Marker | Rank | AUC (95% CI) | Rank | AUC (95% CI) | Rank | AUC (95% CI) |
| Adiponectin | 7 | 0.51 (0.40-0.62) | 5 | 0.64 (0.52-0.74) | 2 | 0.59 (0.44-0.17) |
| PAI | 8 | 0.51 (0.39-0.63) | 10 | 0.53 (0.38-0.66) | 9 | 0.50 (0.37-0.63) |
| Resisitin | 6 | 0.52 (0.42-0.54) | 7 | 0.58 (0.45-0.71) | 8 | 0.52 (0.40-0.65) |
| HGF | 4 | 0.57 (0.47-0.69) | 4 | 0.64 (0.52-0.77) | 12 | 0.48 (0.35-0.61) |
| IL-1 beta | 1 | 0.61 (0.49-0.72) | 2 | 0.66 (0.54-0.78) | 11 | 0.48 (0.36-0.61) |
| IL-6 | 2 | 0.61 (0.49-0.70) | 3 | 0.64 (0.52-0.75) | 10 | 0.49 (0.36-0.61) |
| IL-8 | 3 | 0.59 (0.48-0.70) | 12 | 0.48 (0.35-0.62) | 1 | 0.65 (0.54-0.76) |
| Insulin | 9/10 | 0.50 (0.39-0.61) | 9 | 0.55 (0.41-0.67) | 5 | 0.55 (0.42-0.67) |
| Leptin | 9/10 | 0.50 (0.38-0.60) | 8 | 0.56 (0.43-0.69) | 7 | 0.54 (0.42-0.55) |
| MCP-1 | 12 | 0.49 (0.38-0.60) | 13 | 0.46 (0.33-0.59) | 13 | 0.46 (0.34-0.59) |
| NGF | 5 | 0.55 (0.45-0.66) | 1 | 0.69 (0.57-0.81) | 14 | 0.56 (0.44-0.69) |
| TNF alpha | 13 | 0.47 (0.36-0.59) | 11 | 0.51 (0.38-0.63) | 6 | 0.54 (0.42-0.66) |
| Adipo:Lep | 11 | 0.50 (0.40-0.62) | 6 | 0.61 (0.49-0.73) | 3 | 0.57 (0.46-0.69) |

AUC = area under the receiver operating characteristic curve; PAI = plasminogen activator inhibitor; HGF = hepatocyte growth factor; IL = interleukin; MCP-1 = monocyte chemotactic protein 1; NGF = nerve growth factor, TNF = tumor necrosis factor

TABLE 5
ROC curve analysis of adipokines versus prostate
cancer in patients with a BMI < 27 Kg/m²

| | | | High Grade Cancer vs No | | Low Grade Cancer vs No | |
|---|---|---|---|---|---|---|
| | Cancer versus No Cancer | | High Grade Cancer | | Low Grade Cancer | |
| Marker | Rank | AUC (95% CI) | Rank AUC (95% CI) | | Rank | AUC (95% CI) |
| Adiponectin | 6 | 0.54 (0.41-0.65) | 2 | 0.60 (0.45-0.74) | 7 | 0.55 (0.42-0.67) |
| PAI | 10 | 0.47 (0.36-0.60) | 13 | 0.37 (0.26-0.49) | 2 | 0.60 (0.47-0.74) |
| Resisitin | 1 | 0.64 (0.52-0.74) | 5 | 0.52 (0.39-0.66) | 1 | 0.67 (0.55-0.79) |
| HGF | 7 | 0.53 (0.42-0.65) | 7 | 0.51 (0.37-0.65) | 11 | 0.53 (0.38-0.68) |
| IL-1 beta | 8/9 | 0.50 (0.38-0.62) | 8 | 0.50 (0.39-0.62) | 5 | 0.55 (0.40-0.71) |
| IL-6 | 4/5 | 0.57 (0.45-0.69) | 4 | 0.56 (0.43-0.69) | 9 | 0.54 (0.39-0.69) |
| IL-8 | 3 | 0.58 (0.46-0.69) | 6 | 0.51 (0.39-0.64) | 3 | 0.60 (0.45-0.75) |
| Insulin | 12 | 0.45 (0.35-0.57) | 11 | 0.48 (0.34-0.61) | 8 | 0.54 (0.39-0.68) |
| Leptin | 13 | 0.43 (0.32-0.54) | 12 | 0.45 (0.34-0.59) | 6 | 0.55 (0.42-0.68) |
| MCP-1 | 2 | 0.61 (0.49-0.72) | 1 | 0.61 (0.47-0.73) | 10 | 0.53 (0.38-0.69) |
| NGF | 8/9 | 0.50 (0.39-0.62) | 9 | 0.50 (0.38-0.62) | 13 | 0.48 (0.33-0.63) |
| TNF alpha | 11 | 0.46 (0.35-0.57) | 10 | 0.50 (0.37-0.63) | 4 | 0.56 (0.44-0.69) |
| Adipo:Lep | 4/5 | 0.57 (0.44-0.68) | 3 | 0.58 (0.43-0.72) | 12 | 0.49 (0.36-0.62) |

AUC = area under the receiver operating characteristic curve; PAI = plasminogen activator inhibitor; HGF = hepatocyte growth factor; IL = interleukin; MCP-1 = monocyte chemotactic protein 1; NGF = nerve growth factor, TNF = tumor necrosis factor

TABLE 6
All Patients: Multivariable models Predicting Prostate
Cancer versus No Prostate CancerN = 200, 100 with PCa.

| Model | Variable | OR | 95% CI | | P value |
|---|---|---|---|---|---|
| Model # 1 | African (yes vs. no) | 0.32 | 0.06 | 1.67 | 0.175 |
| Only Clincial | Prev negative Bx | 0.45 | 0.22 | 0.91 | 0.026 |
| Variables Are | (yes vs. no) | | | | |
| Considered | DRE (positive vs. | 2.86 | 1.34 | 6.12 | 0.007 |
| AUC: 0.68 95% | negative) | | | | |
| CI: (0.60, 0.75) | BMI | 1.07 | 1.00 | 1.15 | 0.064 |
| AIC: 255.0 | log PSA | 1.36 | 0.88 | 2.08 | 0.166 |
| Model #2 | Prev negative Bx | 0.45 | 0.21 | 0.96 | 0.040 |
| All Clincial | (yes vs. no) | | | | |
| and Adipokine | DRE (positive vs. | 3.28 | 1.42 | 7.59 | 0.005 |
| Variables are | negative) | | | | |
| Considered | BMI | 1.11 | 1.01 | 1.22 | 0.034 |
| AUC: 0.74 95% | log PSA | 1.37 | 0.87 | 2.14 | 0.173 |
| CI: (0.67, 0.81) | log hgf | 2.16 | 1.16 | 4.03 | 0.016 |
| AIC: 242.0 | log il_6 | 2.01 | 0.97 | 4.17 | 0.061 |
| | log insulin | 0.52 | 0.29 | 0.91 | 0.023 |
| | log leptin | 0.78 | 0.55 | 1.11 | 0.163 |
| | log mcp_1 | 2.17 | 1.34 | 3.52 | 0.002 |
| | log tnf_alpha | 0.36 | 0.13 | 1.00 | 0.050 |
| Model #3 | Prev negative Bx | 0.43 | 0.21 | 0.87 | 0.020 |
| Adipokine Variables | (yes vs. no) | | | | |
| are Removed | DRE (positive vs. | 2.88 | 1.36 | 6.13 | 0.006 |
| from Model #2 | negative) | | | | |
| AUC: 0.66 95% | BMI | 1.06 | 0.99 | 1.14 | 0.094 |
| CI: (0.58, 0.74) | log PSA | 1.36 | 0.88 | 2.09 | 0.162 |
| AIC: | | | | | |

| Comparison | P value |
|---|---|
| Model #1 vs. Model #2 | 0.045 |
| Model #2 vs. Model #3 | 0.015 |
| Model #1 vs. Model #3 | 0.143 |

All Patients: Multivariable models Predicting High Grade
PCa versus Low Grade or No PCa N = 200, 50 with HG PCa.

| Model | Variable | OR | 95% CI | | P value |
|---|---|---|---|---|---|
| Model # 1 | Prev negative Bx | 0.15 | 0.05 | 0.44 | 0.001 |
| Only Clincial | (yes vs. no) | | | | |
| Variables Are | DRE (positive vs. | 5.69 | 2.44 | 13.27 | <0.001 |
| Considered | negative) | | | | |
| AUC: 0.77 95% | log PSA | 3.00 | 1.54 | 5.84 | 0.001 |
| CI: (0.69, 0.85) | | | | | |
| AIC: 179.61 | | | | | |
| Model #2 | Prev negative Bx | 0.14 | 0.04 | 0.44 | <0.001 |
| All Clincial | (yes vs. no) | | | | |
| and Adipokine | DRE (positive vs. | 6.65 | 2.73 | 16.16 | <0.001 |
| Variables are | negative) | | | | |
| Considered | log PSA | 3.83 | 1.83 | 8.02 | <0.001 |
| AUC: 0.81 95% | log il 8 | 0.77 | 0.42 | 1.41 | 0.395 |
| CI: (0.74, 0.88) | log ngf | 1.66 | 0.94 | 2.92 | 0.080 |
| AIC: 173.1 | | | | | |
| Model #3 = | | | | | |
| Model #1 | | | | | |

| Comparison | P value |
|---|---|
| Model #1 vs. Model #2 | 0.058 |

Models were restricted to a maximum of 5 predictors to avoid overfitting because the data only have 50 observations with HG PCa.

### REFERENCE LIST

1. Hsing, A. W., Sakoda, L. C., Chua, S., Jr.: Obesity, metabolic syndrome, and prostate cancer. Am J Clin Nutr, 86: s843, 2007
2. Amling, C. L.: Relationship between obesity and prostate cancer. Curr Opin Urol, 15: 167, 2005
3. Freedland, S. J., Aronson, W. J.: Examining the relationship between obesity and prostate cancer. Rev Urol, 6: 73, 2004
4. Gong, Z., Neuhouser, M. L., Goodman, P. J. et al.: Obesity, diabetes, and risk of prostate cancer: results from the prostate cancer prevention trial. Cancer Epidemiol Biomarkers Prev, 15: 1977, 2006
5. Rodriguez, C., Freedland, S. J., Deka, A. et al.: Body mass index, weight change, and risk of prostate cancer in the Cancer Prevention Study II Nutrition Cohort. Cancer Epidemiol Biomarkers Prev, 16: 63, 2007
6. Rohrmann, S., Roberts, W. W., Walsh, P. C. et al.: Family history of prostate cancer and obesity in relation to high-grade disease and extraprostatic extension in young men with prostate cancer. Prostate, 55: 140, 2003
7. Mistry, T., Digby, J. E., Desai, K. M. et al.: Obesity and prostate cancer: a role for adipokines. Eur Urol, 52: 46, 2007
8. Renehan, A. G., Roberts, D. L., Dive, C.: Obesity and cancer: pathophysiological and biological mechanisms. Arch Physiol Biochem, 114: 71, 2008
9. Baillargeon, J., Platz, E. A., Rose, D. P. et al.: Obesity, adipokines, and prostate cancer in a prospective population-based study. Cancer Epidemiol Biomarkers Prev, 15: 1331, 2006
10. Chang, S., Hursting, S. D., Contois, J. H. et al.: Leptin and prostate cancer. Prostate, 46: 62, 2001
11. Saglam, K., Aydur, E., Yilmaz, M. et al.: Leptin influences cellular differentiation and progression in prostate cancer. J Urol, 169: 1308, 2003
12. Freedland, S. J., Sokoll, L. J., Mangold, L. A. et al.: Serum leptin and pathological findings at the time of radical prostatectomy. J Urol, 173: 773, 2005
13. Freedland, S. J., Sokoll, L. J., Platz, E. A. et al.: Association between serum adiponectin, and pathological stage and grade in men undergoing radical prostatectomy. J Urol, 174: 1266, 2005
14. Housa, D., Vernerova, Z., Heracek, J. et al.: Adiponectin as a potential marker of prostate cancer progression: studies in organ-confined and locally advanced prostate cancer. Physiol Res, 57: 451, 2008
15. Adler, H. L., McCurdy, M. A., Kattan, M. W. et al.: Elevated levels of circulating interleukin-6 and transforming growth factor-beta1 in patients with metastatic prostatic carcinoma. J Urol, 161: 182, 1999
16. Drachenberg, D. E., Elgamal, A. A., Rowbotham, R. et al.: Circulating levels of interleukin-6 in patients with hormone refractory prostate cancer. Prostate, 41: 127, 1999
17. Nakashima, J., Tachibana, M., Horiguchi, Y. et al.: Serum interleukin 6 as a prognostic factor in patients with prostate cancer. Clin Cancer Res, 6: 2702, 2000
18. Babaian, R. J., Toi, A., Kamoi, K. et al.: A comparative analysis of sextant and an extended 11-core multisite directed biopsy strategy. J Urol, 163: 152, 2000
19. Pepe, M. S., Etzioni, R., Feng, Z. et al.: Phases of biomarker development for early detection of cancer. J Natl Cancer Inst, 93: 1054, 2001
19. Parekh, D. J., Ankerst, D. P., Baillargeon, J. et al.: Assessment of 54 biomarkers for biopsy-detectable prostate cancer. Cancer Epidemiol Biomarkers Prev, 16: 1966, 2007
20. Shariat, S. F., Walz, J., Roehrborn, C. G. et al.: External validation of a biomarker-based preoperative nomogram predicts biochemical recurrence after radical prostatectomy. J Clin Oncol, 26: 1526, 2008
21. Gupta, A., Karakiewicz, P. I., Roehrborn, C. G. et al.: Predictive value of plasma hepatocyte growth factor/scatter factor levels in patients with clinically localized prostate cancer. Clin Cancer Res, 14: 7385, 2008
22. Nagakawa, O., Yamagishi, T., Fujiuchi, Y. et al.: Serum hepatocyte growth factor activator (HGFA) in benign prostatic hyperplasia and prostate cancer. Eur Urol, 48: 686, 2005
23. Naughton, M., Picus, J., Zhu, X. et al.: Scatter factor-hepatocyte growth factor elevation in the serum of patients with prostate cancer. J Urol, 165: 1325, 2001
24. Nishimura, K., Arichi, N., Tokugawa, S. et al.: Hepatocyte growth factor and interleukin-6 in combination with prostate volume are possible prostate cancer tumor markers in patients with gray-zone PSA levels. Prostate Cancer Prostatic Dis, 11: 258, 2008
25. Arrighi, N., Bodei, S., Zani, D. et al.: Nerve growth factor signaling in prostate health and disease. Growth Factors, 28: 191
26. Sigala, S., Tognazzi, N., Rizzetti, M. C. et al.: Nerve growth factor induces the re-expression of functional androgen receptors and p75(NGFR) in the androgen-insensitive prostate cancer cell line DU145. Eur J Endocrinol, 147: 407, 2002
27. Sigala, S., Bodei, S., Missale, C. et al.: Gene expression profile of prostate cancer cell lines: effect of nerve growth factor treatment. Mol Cell Endocrinol, 284: 11, 2008
28. Ricote, M., Garcia-Tunon, I., Bethencourt, F. R. et al.: Interleukin-1 (IL-1alpha and IL-1 beta) and its receptors (IL-1RI, IL-1RII, and IL-1Ra) in prostate carcinoma. Cancer, 100: 1388, 2004
29. Voronov, E., Shouval, D. S., Krelin, Y. et al.: IL-1 is required for tumor invasiveness and angiogenesis. Proc Natl Acad Sci USA, 100: 2645, 2003
30. Kim, H. J., Lee, Y. S., Won, E. H. et al.: Expression of resistin in the prostate and its stimulatory effect on prostate cancer cell proliferation. BJU Int
31. Housa, D., Vernerova, Z., Heracek, J. et al.: Serum resistin levels in benign prostate hyperplasia and non-metastatic prostate cancer: possible role in cancer progression. Neoplasma, 55: 442, 2008
32. Veltri, R. W., Miller, M. C., Zhao, G. et al.: Interleukin-8 serum levels in patients with benign prostatic hyperplasia and prostate cancer. Urology, 53: 139, 1999
33. Uehara, H., Troncoso, P., Johnston, D. et al.: Expression of interleukin-8 gene in radical prostatectomy specimens is associated with advanced pathologic stage. Prostate, 64: 40, 2005
34. Araki, S., Omori, Y., Lyn, D. et al.: Interleukin-8 is a molecular determinant of androgen independence and progression in prostate cancer. Cancer Res, 67: 6854, 2007

1. An assay for of qualifying/predicting the likelihood of prostate cancer in a subject comprising:
a) categorizing the subject as high BMI (>27) or low BMI (≦27); and
b) measuring the level of expression of at least one adipokine in a serum sample from the subject;
c) correlating the level of expression of the at least one adipokine in the serum sample to a control sample; and
d) determining that the level of expression is predictive for prostate cancer if there exists a differential level of expression between the serum sample and the control sample;
wherein the at least one adipokine is at least one of:
i. Resistin, MCP-1 and IL-8 if the subject is low BMI; and
ii. IL-1β, IL-6 and IL-8 if the subject is high BMI.

2. The assay of claim 1, wherein the level of expression of the at least one adipokine is measured by measuring the protein level in the serum sample.

3. The assay of claim 1, wherein the at least one adipokine is Resistin if the subject is low BMI.

4. The assay of claim 1, wherein the differential level of expression predictive of prostate cancer is characterized by a higher level of expression in the serum sample than in the control sample.

5. An assay for qualifying/predicting the likelihood of low-grade prostate cancer in a subject comprising:
a) categorizing the subject as high BMI (>27) or low BMI (≦27); and
b) measuring the level of expression of at least one adipokine in a serum sample from the subject;
c) correlating the level of expression of the at least one adipokine in the serum sample to a control sample; and
d) determining that the level of expression is predictive for low-grade prostate cancer if there exists a differential level of expression between the serum sample and the control sample;
wherein the at least one adipokine is at least one of:
i. Resistin, PAI and IL-8 if the subject is low BMI; and
ii. IL-8, Adipo:leptin and Adiponectin if the subject is high BMI.

6. The assay of claim 5, wherein the level of expression of the at least one adipokine is measured by measuring the protein level in the serum sample.

7. The assay of claim 5, wherein the at least one adipokine is Resistin if the subject is low BMI.

8. The assay of claim 5, wherein the at least one adipokine is IL-8 if the subject is high BMI.

9. The assay of claim 5, wherein the differential level of expression predictive of prostate cancer is characterized by a higher level of expression in the serum sample than in the control sample.

10. An assay for qualifying/predicting the likelihood of high-grade prostate cancer in a subject comprising:
a) categorizing the subject as high BMI (>27) or low BMI (≦27); and
b) measuring the level of expression of at least one adipokine in a serum sample from the subject;
c) correlating the level of expression of the at least one adipokine in the serum sample to a control sample; and
d) determining that the level of expression is predictive for high-grade prostate cancer if there exists a differential level of expression between the serum sample and the control sample;
wherein the at least one adipokine is at least one of:
i. MCP-1, Adipo:leptin and Adiponectin if the subject is low BMI; and
ii. NGF, IL-1β, IL-6, HGF and Adiponectin if the subject is high BMI.

11. The assay of claim 10, wherein the level of expression of the at least one adipokine is measured by measuring the protein level in the serum sample.

12. The assay of claim 10, wherein the differential level of expression predictive of prostate cancer is characterized by a higher level of expression in the serum sample than in the control sample.
